(19) United States
(12) Patent Application Publication
(10) Pub. No.: US 2026/0298947 A1
(43) Pub. Date: Oct. 1, 2026

(54) METHOD FOR THE IN VITRO DIAGNOSIS OF A NEURODEGENERATIVE DISEASE

(71) Applicants: UNIVERSITE GRENOBLE ALPES, Saint Martin D'heres (FR); INSTITUT NATIONAL DE LA SANTE ET DE LA RECHERCHE MEDICALE, Paris (FR)

(72) Inventors: Muriel-Réjane JACQUIER-SARLIN, Saint Martin Le Vinoux (FR); Alain BUISSON, Montbonnt (FR)

(73) Assignees: UNIVERSITE GRENOBLE ALPES, Saint Martin D'heres (FR); INSTITUT NATIONAL DE LA SANTE ET DE LA RECHERCHE MEDICALE, Paris (FR)

(21) Appl. No.: 19/479,469

(22) PCT Filed: May 3, 2024
(86) PCT No.: PCT/FR2024/050583

§ 371 Date: Oct. 28, 2025

(30) Foreign Application Priority Data
May 5, 2023 (FR) FR2304537

(51) Int. Cl. G01N 33/68

(52) U.S. Cl. CPC G01N 33/6896; G01N 2333/4709; G01N 2440/14; G01N 2500/02; G01N 2800/2821; G01N 2800/52

(57) ABSTRACT

A method for the in vitro diagnosis of a neurodegenerative disease, in a human or animal individual, at an early stage, including the step which includes in detecting the presence of at least one marker chosen from the derived forms of amyloid beta (Aβ) peptides selected from the oligomers of said peptides and the prefibrillar and fibrillar aggregated forms of said peptides, and the derived forms of phosphorylated tau proteins selected from the hyperphosphorylated forms of said proteins, the aggregated forms of said proteins and the modified phosphorylated tau proteins resulting from one or several post-translational modifications, the presence of the marker(s) being detected in a stool sample of said individual.

The present invention concerns the detection of neurodegenerative diseases (NDDs), and more specifically the detection of these diseases at an early stage.

By neurodegenerative disease, is meant a chronic, progressive disease that affects the nervous system and leads to irreversible cognitive, behavioral, sensory, and motor disorders. Alzheimer's disease is the most common form of dementia in humans, it accounts for 60 to 70% of dementia cases, followed by Parkinson's disease in second place, and then diseases such as Charcot's disease and Huntington's disease. They are all characterized by a progressive loss of neuron function and by a major risk factor: aging.

Several neurodegenerative diseases, and in particular the aforementioned diseases, are classified as proteinopathies, in that they are associated with the presence of proteins in an abnormally aggregated and/or misfolded form, the accumulation of which in the brain leads to toxicity. Belonging to this group of pathologies are tauopathies which have in common an abnormal accumulation of tau proteins in the form of clusters and which share clinical and pathological similarities. The invention relates to the diagnosis of such diseases and particularly those associated with at least one of the amyloid beta and tau proteins.

The present invention is described with reference to tauopathies, in particular Alzheimer's disease, but it is not limited thereto and extends to all NDDs meeting the above definition.

Alzheimer's disease (AD) is an incurable neurodegenerative disease that begins with a long asymptomatic phase and that is characterized by a progressive decline in cognitive capacities accompanied by neuronal and synaptic loss.

There is currently no curative treatment for Alzheimer's disease and the failure of recent therapeutic strategies is largely the result of too late diagnosis of the disease when the first characteristic signs are already visible.

The diagnosis of AD has, however, evolved considerably in recent years with the identification of the concomitant presence of two types of brain injuries that are characteristic of the disease: extracellular deposits of amyloid peptides (Aβ), in particular Aβ42, designated as amyloid plaques or senile plaques, and intraneuronal deposits of hyperphosphorylated tau proteins (P-tau), associated with neurofibrillary degeneration (NFD). Thus, to date, the detection of AD is based on neuropsychological tests, the assay of amyloid peptides and hyperphosphorylated tau proteins in the cerebrospinal fluid (CSF), morphological imaging (MRI) and positron emission tomography (PET) technology to detect functional tracers (radioactive fluorodeoxyglucose or ¹⁸ FDG PET) or molecular tracers (amyloid-PET and tau-PET).

Detection of AD markers, Aβ and tau, is performed in CSF because it contains proteins from the brain, but CSF collection can be difficult depending on the patient's age and health status since it requires a lumbar puncture, an invasive procedure.

The interest of these markers lies in their synergistic use to refine the positive diagnosis and begin providing prognostic information to the patient. However, even if they are very sensitive and specific for the positive diagnosis and for detecting pathophysiological injuries from the stage of memory complaints or minor cognitive disorders, well before cognitive decline and loss of autonomy, the diagnosis occurs in the clinical phase of AD, which is preceded by a preclinical phase lasting about 20 years or more, during which no or only subtle symptoms of the disease are observed. The identification of markers in this phase of the disease would allow:
1) delaying the onset of the disease or even preventing AD dementia; indeed, current treatments can only help improve the clinical symptoms, but cannot delay or reverse the progression of AD;
2) earlier intervention of therapeutic strategies that could have greater effectiveness; and
3) systematic monitoring of patients and the effectiveness of treatments.

Studies have been conducted on detecting a molecular signature of AD in a body fluid such as blood or saliva, which can be collected in a minimally invasive manner.

Regarding the Aβ marker, it is produced in the brain and is indeed present in blood plasma. However, it is also produced in platelets and peripheral tissues, making it difficult to distinguish between brain-derived and produced Aβ in plasma. In addition, plasma concentrations of Aβ42 are at least 100 times lower than in CSF (typically, in the range of a few pg/ml) and are diluted by the high concentration of other non-specific protein components. Finally, plasma Aβ peptides, due to their hydrophobic nature, tend to bind to erythrocytes and other plasma proteins, which may lead to masking of epitopes and thus prevent detection of peptides.

As for the P-tau marker, secreted in CSF, it is also present in blood plasma. A semi-sensitive test for this marker phosphorylated on threonine 181 (similar to the most widely used CSF test) with electrochemiluminescence detection has been developed. This test made it possible to demonstrate that the plasma concentration of P-tau was higher in patients suffering from Alzheimer's dementia than in controls (2 to 4 times higher). This increase appears in the preclinical phase of the disease but can also occur episodically in other circumstances such as acute hypoxic brain injuries in the case of concussion and during anesthesia, which has led to the rejection of this test.

Other candidate proteins in blood have been found by the proteomic approach (Kitamura Y. et al., Plasma protein profiling for potential biomarkers in the early diagnosis of Alzheimer's disease, Neurological Research. Taylor & Francis, 39(3), pp 231-238, (2017) but they did not make it possible to achieve satisfactory results.

Document WO2019/192969A1 describes a method for the diagnosis or prognosis, at a so-called early stage, of a disease associated with an amyloid peptide or protein in a biological sample, consisting in specifically detecting aggregates of this peptide or protein therein, or determining their number, concentration or size. A biological sample may be a sample of blood, urine, synovial fluid, stool or saliva. This method may be supplemented by a step consisting in detecting, in said sample, the tau protein, which is possibly phosphorylated or determining its number, concentration or size.

Document WO2021/239700A2 discloses a method for detecting oligomers and aggregated forms of the Aβ peptide as pathological markers of a neurodegenerative disease, in particular Alzheimer's disease, in a stool sample, the method making it possible to determine the number, concentration and size of the aggregates. The choice of stool (or feces) considered as a reflection of the presence of these markers in the brain, cerebrospinal fluid and blood, spares the individual invasive sampling and the need for health care professionals to perform the sampling. This method is applicable to the diagnosis, in particular early diagnosis, of Alzheimer's disease.

The accumulation of oligomeric and aggregated forms of amyloid peptides and phosphorylated forms of tau proteins are indeed markers of a neurodegenerative disease and the more specific their detection is compared to monomeric forms and the more sensitive it is, the earlier the diagnosis is made. But, as previously indicated, the preclinical phase is very long and this diagnosis occurs late, these aggregated forms being already ubiquitous and the clinical phase of the disease has begun.

To date, there is still a crucial need to identify viable markers for AD that monitor disease progression over time and the concentrations of which can change in response to a treatment of the disease.

Surprisingly, it has been discovered that a viable and reliable molecular signature of AD is present in the stool (or feces) of a human or animal, at an early stage. These markers are amyloid beta (Aβ) peptides and phosphorylated tau proteins (P-tau) and their respective derived forms, variations of which make it possible to characterize an early stage of neurodegenerative disease of the proteinopathy type such as a tauopathy, and in particular Alzheimer's disease.

Thus, the invention concerns a method for the in vitro diagnosis of a neurodegenerative disease, in a human or animal individual, comprising the step which consists in detecting the presence of at least one marker chosen from the derived forms of amyloid beta (Aβ) peptides selected from the oligomers of said peptides and the prefibrillar and fibrillar aggregated forms of said peptides; and the derived forms of phosphorylated tau proteins (P-tau) selected from the hyperphosphorylated forms of said proteins, the aggregated forms of said proteins and the modified phosphorylated tau proteins resulting from one or several post-translational modifications, the presence of the marker(s) being detected in a stool (or feces) sample of said individual.

According to one implementation of the method of the invention, the neurodegenerative disease is a tauopathy, and the presence of at least one marker chosen from said derived forms of phosphorylated tau proteins is detected in a stool sample of said individual. According to another implementation of the method of the invention, the presence of at least one marker chosen from said derived forms of amyloid beta peptides and at least one marker chosen from said derived forms of phosphorylated tau proteins is determined.

In all the objects of the invention described in the present text, by the expression “at least one marker chosen from the derived forms of amyloid beta (Aβ) peptides and the derived forms of phosphorylated tau proteins (P-tau)”, it should be understood that said object involves one or several markers chosen indifferently from the derived forms of amyloid beta (Aβ) peptides and the derived forms of phosphorylated tau proteins (P-tau). When it involves several markers, any combination thereof can be envisaged.

The detection of these markers in stool, which has the advantage of being less invasive and less expensive than their detection in CSF, is also reproducible and sensitive. Unexpectedly, it has proven to be specific for the early diagnosis of AD, and even capable of revealing subjects at risk of developing AD. In addition, the biomarkers analyzed after sampling have proved to be stable over time, and their concentration does not vary up to 7 days post-sampling, indicating that there is little, if any, degradation of the sample during this period. This is particularly the case for the tau protein and its derived forms, which degrade rapidly in a liquid biological sample, in particular aqueous sample, such as blood or serum (Schöll et al., Biomarkers for tau pathology, Molecular and Cellular Neuroscience, 97, pp 18-33, 2019) and (Randall et al., Tau proteins in serum predict neurological outcome after hypoxic brain injury from cardiac arrest: results of a pilot study. Resuscitation 84, pp 351-356, 2013). The authors have in fact observed that, in the stool, these markers can be detected several days after sampling, even a week, without any change over time, either in their structure or in their concentration. Given the practicality of stool sampling that can be carried out by the individual himself and then deposited or sent to a laboratory for analysis, this discovery is a real progress in the diagnosis of tauopathies.

The present invention makes a real contribution to the diagnosis of neurodegenerative diseases, such as Alzheimer's disease.

As mentioned above, amyloid beta (Aβ) peptides and phosphorylated tau proteins as well as their respective derived forms are a group of known and well-identified molecules. Their definitions are recalled below.

By amyloid beta (Aβ) peptide is meant a structure of 38 to 42 amino acids resulting from the enzymatic cleavage of the amyloid precursor protein (APP) involving beta- or gamma-secretase enzymes. The gene encoding this protein is located on chromosome 21. Amyloid beta peptide, which is soluble, has the ability to oligomerize into soluble oligomers of various sizes and to aggregate to form prefibrillar structures and fibrillar amyloid plaques, which are insoluble. Thus, by derived form of amyloid beta peptide is meant the oligomers of amyloid beta peptide of any size, and the aggregated prefibrillar and fibrillar forms (Chen, Gf., et al. Amyloid beta: structure, biology and structure-based therapeutic development. Acta Pharmacol Sin 38, 1205-1235 (2017). It should be noted that, according to a particular implementation of the invention, namely detection by DotBlot, these different conformations can be distinguished. This is an additional advantage for the invention, as the oligomeric forms, which are not yet aggregated, are the most toxic. The definition of derived form of the Aβ peptide also includes modified peptides resulting from one or several post-translational modifications such as a truncated Aβ peptide.

Tau protein is a protein encoded by the microtubule-associated protein tau (MAPT) gene on human chromosome 17 and is described in Xia et al. Molecular Neurodegeneration 16, 37 (2021 ). It is a water-soluble protein, it has approximately 80 potential phosphorylation sites, on the amino acids serine and threonine. In a healthy individual, some of these sites are phosphorylated under physiological conditions, but in certain pathological conditions, and in particular in NDDs, the protein is highly phosphorylated (designated as P-tau, phosphorylated tau protein or hyperphosphorylated tau protein). According to the invention, by phosphorylated tau protein is meant an abnormal phosphorylation of some previously identified key sites of the tau protein. By derived form of phosphorylated tau protein, is meant the hyperphosphorylated forms found on pathological sites, aggregated tau proteins and modified proteins resulting from one or several post-translational modifications such as a truncated tau protein.

The derived forms of the Aβ peptide or the tau protein resulting from post-translational modification(s) are a concept known to those skilled in the art. For example, reference may be made to the following articles:

Carroll T, Guha S, Nehrke K and Johnson G V W; Tau Post-Translational Modifications: Potentiators of Selective Vulnerability in Sporadic Alzheimer's Disease. Biology 2021, 10, 1047;

Alquezar C, Arya S and Kao A W; Tau Post-translational Modifications: Dynamic Transformers of Tau Function, Degradation, and Aggregation. Front. Neurol. 2021, 11:595532; and

Barykin E P, Mitkevich V A, Kozin S A and Makarov A A; Amyloid B Modification: A Key to the Sporadic Alzheimer's Disease? Front. Genet. 2017, 8:58.

The Aβ peptide and the tau protein are water-soluble entities; as previously indicated, they tend to form oligomers which, as they increase in size, become less and less water-soluble, evolving into aggregates which are insoluble in an aqueous medium.

As a marker chosen from amyloid beta (Aβ) peptides and phosphorylated tau proteins (P-tau), as well as their respective derived forms, it is meant that they are in the free state, or in a state combined with one or several other entities such as other proteins/peptides, not preventing their detection.

In a variant of the invention, amyloid beta (Aβ) peptides and phosphorylated tau proteins (P-tau), as non-specific monomers for an early diagnosis of a neurodegenerative disease, can be used as marker(s) in calculating a ratio of derived forms to all forms of said peptide or said protein; all forms including peptides (Aβ) and derived forms thereof as previously described or phosphorylated tau proteins (P-tau) and derived forms thereof as previously described, respectively.

According to a preferred implementation of each of the methods, kits and uses of the invention, at least one marker chosen from said derived forms of amyloid beta peptides, and at least one marker chosen from said derived forms of phosphorylated tau proteins, are detected.

By early stage according to the invention, is meant a stage at which mild symptoms of dementia have not yet appeared or appear, without a direct link having been established with a neurodegenerative disease. For example, it may correspond to the stage at which the first memory disorders are detected or at which other disorders are observed, in particular during screening campaigns such as those implemented for cancers from the age of 50. The authors also discovered that at this early stage, said derived forms of amyloid beta peptides and said derived forms of phosphorylated tau proteins are not accumulated in the brain. This means that at this early stage, the brain is still capable of eliminating these derived forms; the latter may therefore be present there but not in a pathological manner since they do not accumulate there. It appears that at this early stage according to the invention, biological tissues and fluids do not contain the same forms of molecules, monomeric and soluble, or aggregated and insoluble. Compared to the prior art and in particular compared to the aforementioned documents, WO2019/192969A1 and WO2021/239700A2, the invention is based on the identification of a process for eliminating the derived forms of Aβ peptides and tau proteins produced in the brain, in the stool where they can be detected before their toxic accumulation in the brain.

The presence of these derived forms in the brain can be determined by medical imaging such as positron emission tomography (PET); those skilled in the art can refer to the following documents:

Brain imaging using PET and radiotracers will reflect reliably on fibrillar deposits of amyloid in the brain that are >1 mm in size (20-22) 20. Saint-Aubert, L., Lemoine, L., Chiotis, K., Leuzy, A., Rodriguez-Vieitez, E., and Nordberg, A. (2017) Molecular neurodegeneration 12, 19

Garibotto, V., Herholz, K., Boccardi, M., Picco, A., Varrone, A., Nordberg, A., Nobili, F., Ratib, O., and Geneva Task Force for the Roadmap of Alzheimer's, B. (2017) Neurobiology of aging 52, 183-195

Chiotis, K., Saint-Aubert, L., Boccardi, M., Gietl, A., Picco, A., Varrone, A., Garibotto, V., Herholz, K., Nobili, F., Nordberg, A., and Geneva Task Force for the Roadmap of Alzheimer's, B. (2017) Neurobiology of aging 52, 214-227.

It was further found that said derived forms of Aβ peptides and phosphorylated tau proteins detected in the stool are different from those present in the intestine, suggesting that they reach the stool by a route other than the digestive tract. This is corroborated by the study described in A. Lionnet et al, Acta Neuropathologica Communications (2018) 6:65.

In one variant, the method of the invention comprises the following steps:
said stool sample of said individual is brought into contact with at least one antibody or antibody portion specifically recognizing at least one of said markers, under conditions allowing the formation of a complex between said antibody or antibody portion and said marker, and
the presence of said complex, where appropriate, the presence of said complex determining the individual's involvement by a neurodegenerative disease is detected.

According to a variant of the invention, this method allows the in vitro diagnosis of Alzheimer's disease in an individual.

As previously mentioned, one of the interests of the invention is to obtain an early diagnosis. In this respect, the tested individual may be asymptomatic or has some minor cognitive disorders that may not be directly associated with NDD.

The invention also concerns a method for monitoring the condition of a human or animal individual suspected of suffering from a neurodegenerative disease, comprising the following steps:
a first stool sample of said individual is brought into contact with at least one antibody or antibody portion specifically recognizing at least one marker chosen from amyloid beta peptides, the derived forms of said peptides, phosphorylated tau proteins and the derived forms of said proteins, under conditions allowing the formation of a complex between said antibody or antibody portion and said marker,
the content of said complex in the first sample is measured,
then a second stool sample of said individual, said second sample being taken after the first sample, is brought into contact with said antibody or antibody portion, under conditions allowing the formation of a complex between said antibody or antibody portion and said marker,
the content of said complex in the second sample is measured, a variation between the contents of said complex in the first sample and in the second sample indicating a progression of the disease or a regression of the disease.

In a variant of this method,
the antibody or antibody portion specifically recognizes at least one amyloid beta peptide or a derived form of an amyloid beta peptide and forms a complex therewith, and
a content of said complex in the second sample higher than the content of the same complex in the first sample indicates a progression of the disease, at an early stage, or
a content of said complex in the second sample lower than the content of this same complex in the first sample indicates a regression of the disease.

According to another variant of a method for monitoring the condition of an individual, which can be combined with the or a previous one,
the antibody or antibody portion specifically recognizes at least one soluble phosphorylated tau protein or a derived form of a phosphorylated tau protein and forms a complex therewith, and
a content of said complex in the second sample higher than the content of the same complex in the first sample indicates a progression of the disease, at an early stage, or
a content of said complex in the second sample lower than the content of this same complex in the first sample indicates a regression of the disease.

The method for monitoring the condition of a human or animal individual suspected of suffering from a neurodegenerative disease as shown above is preferably applied at an early stage of the disease up to a stage where the individual has disorders which have just been associated with NDD, as opposed to a late stage of the disease. It has indeed been observed that at a late stage of the disease, an individual may experience alterations in the elimination of plaques, which results in a decrease in the content of the invention's markers in the stool, which, at this stage, should not be interpreted as a regression of the disease.

Any one of the above variants of a monitoring method may be implemented on a stool sample of an individual being treated for said disease, making it possible, in particular, to assess the effectiveness of the treatment.

Thus, the invention also relates to a method for screening a drug for treating a neurodegenerative disease, in an individual suspected of suffering from such a disease, comprising the following steps:

A first stool sample of said individual is brought into contact with at least one antibody or antibody portion specifically recognizing at least one marker chosen from amyloid beta peptides, the derived forms of said peptides, phosphorylated tau proteins and the derived forms of said proteins, under conditions allowing the formation of a complex between said antibody or antibody portion and said marker,
the content of a said complex in the first sample is measured,
then a second stool sample of said individual, said second sample being taken after the first sample, is brought into contact with said antibody or antibody portion, under conditions allowing the formation of a complex between said antibody or antibody portion and said marker,
the content of said complex in the second sample is measured, a variation between the contents of said complex in the first sample and of said complex in the second sample indicating the effectiveness or ineffectiveness of said drug.

In a variant of this method,
the antibody or antibody portion specifically recognizes at least one amyloid beta peptide or a derived form of an amyloid beta peptide and forms a complex therewith, and
the content of said complex in the second sample lower than the content of this complex in the first sample indicates an effectiveness of the drug.

In another variant of the method, which may be combined with a or the above variant,
the antibody or antibody portion specifically recognizes at least one phosphorylated tau protein or a derived form of a phosphorylated tau protein and forms a complex therewith, and
a content of said complex in the second sample lower than the content of this complex in the first sample indicates an effectiveness of the drug.

The time between the collection of the first and second samples will vary depending on the implemented method. Thus, in a method for monitoring the condition of an individual undergoing therapeutic treatment, this time may be short and the method repeated more frequently than in a diagnosis method. Nevertheless, since the method is non-invasive, it is possible to take multiple samples for individualized and close monitoring.

According to any one of the above variants, the non-specific monomeric forms of Aβ peptides and/or P-tau proteins, as non-specific monomers for an early diagnosis of a neurodegenerative disease, may be used as marker(s) in the calculation of the ratio of derived forms of said Aβ peptides or said P-tau proteins/all forms of said Aβ peptides or said P-tau proteins.

The invention further relates to a kit for the in vitro detection of a neurodegenerative disease, at the early stage, of a human or animal individual, comprising
at least one antibody or antibody portion specifically recognizing at least one marker chosen from the derived forms of amyloid beta (Aβ) peptides selected from the oligomers of said peptides and the prefibrillar and fibrillar aggregated forms of said peptides; and from the derived forms of phosphorylated tau proteins selected from the hyperphosphorylated forms of said proteins, the aggregated forms of said proteins and the modified phosphorylated tau proteins resulting from one or several post-translational modifications, and
the means allowing the extraction, from a stool sample of said individual, of at least one marker chosen from amyloid beta (Aβ) peptides, the derived forms, phosphorylated tau proteins and the derived forms of said proteins, and the formation of a complex between said antibody or antibody portion and said marker,

The kit of the invention may also comprise antibodies recognizing the monomeric forms of Aβ peptides and/or P-tau proteins, that are non-specific for an early diagnosis of a neurodegenerative disease, these being able to be used as marker(s) in the calculation of the ratio of derived forms of said Aβ peptides/total quantities of said Aβ peptides and their derived forms, or of the ratio of derived forms of said P-tau proteins/total quantities of said P-tau proteins and their derived forms.

The invention also relates to the use of a marker chosen from the derived forms of amyloid beta (Aβ) peptides selected from the oligomers of said peptides and the prefibrillar and fibrillar aggregated forms of said peptides; and from the derived forms of phosphorylated tau proteins selected from the hyperphosphorylated forms of said proteins, the aggregated forms of said proteins and the modified phosphorylated tau proteins resulting from one or several post-translational modifications, for the in vitro diagnosis of a neurodegenerative disease, in an individual, said marker being detected in the stool of said individual. According to one use of the invention, the diagnosis is performed at an early stage of the disease. In particular, it is intended for the in vitro diagnosis of Alzheimer's disease.

The detection of the derived forms of Aβ peptides and phosphorylated tau proteins can be carried out by techniques within the reach of those skilled in the art. Thus, the AT8 antibody making it possible to specifically detect hyperphosphorylated tau protein. There are anti-toxic oligomeric amyloid peptide (such as Aβo, E22P), anti-fibrillar amyloid peptide (such as Aβ-F, OC) antibodies; the use of these antibodies is illustrated in the following examples.

The present invention is illustrated in the following example and with the support of the figures according to which:

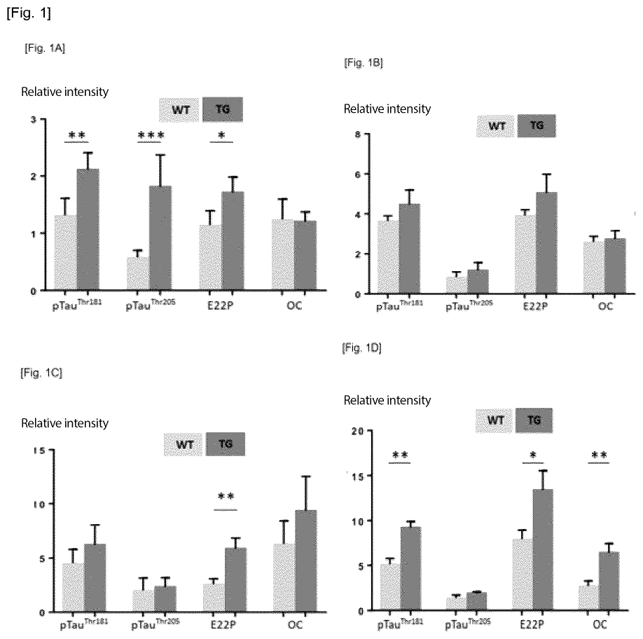

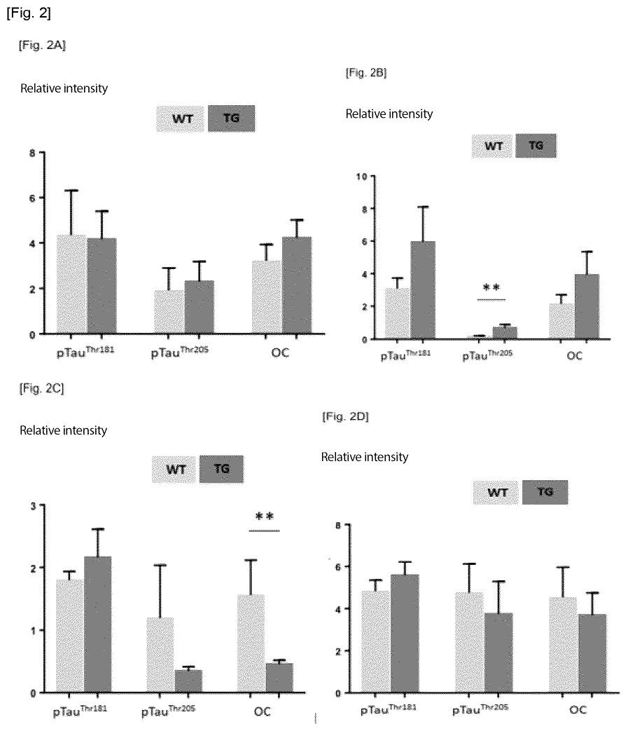

FIG. 1 and FIG. 2 represent the quantity, determined by densitometric analysis and expressed as relative intensity, of toxic oligomeric amyloid peptides (E22P), prefibrillar amyloid peptides (OC) and P-tau proteins (Tau-P^{Thr181}, Tau-P P^{Thr205}) of a fraction of soluble peptides and proteins FIG. 1 and of a fraction of insoluble peptides and proteins FIG. 2, that are extracted from feces of wild-type (WT) mice and AD transgenic mice (APP/PS1-21), as a function of time expressed in months: at 1 month, FIG. 1A and FIG. 2A; at 4 months, FIG. 1B and FIG. 2B; at 6 months, FIG. 1C and FIG. 2C; at 9 months, FIG. 1D and FIG. 2D.

### EXAMPLE

In a transgenic (TG) mouse model of Alzheimer's disease (AD) (APP/PS1-21 mice), the temporal progression (from 1 to 9 months) of AD was monitored in feces, brain, and intestines, respectively.

### 1) Monitoring in Stool

Feces from wild-type (WT) mice and APP/PS1 mice were collected and proteins from these samples were extracted.

### Procedure

### 1.1) Stool Recovery

Mice are placed in a clean cage for two hours, after which the droppings are collected and counted before being frozen at −80° C. Then the samples are thawed, 5-6 droppings (100 mg) are transferred into a tube and placed overnight in an incubator at 56° C. to be dehydrated. The dry material is weighed again in order to determine the water content (%), which partly reflects intestinal function.

### 1.2) Protein Extraction

The dried stool is taken up in 1 ml of RIPA buffer (50 mM Tris-HCl pH 8, 150 mM NaCl, 1% NP-40, 0.5% sodium deoxycholate and 0.1% SDS) containing a cocktail of protease and phosphatase inhibitors. The homogenate is incubated for 10 minutes on ice before being subjected to 5 sonication cycles under the following conditions: 10 seconds of sonication (Amplitude 45)+10 seconds in ice. The homogenate is then centrifuged for 10 minutes at 4° C. at 14,000 rpm. The supernatant is recovered in a clean tube. It contains the soluble proteins.

The pellet is resuspended in 1 mL of 5M guanidine HCl/50 mM Tris-HCl pH 8 buffer containing a cocktail of protease and phosphatase inhibitors. The homogenate is again subjected to 5 sonication cycles under the following conditions: 10 seconds of sonication (Amplitude 45)+10 seconds in ice. The homogenate is then centrifuged for 10 minutes at 4° C. at 14,000 rpm. The supernatant is recovered in a clean tube. It contains the insoluble proteins.

The protein content of each fraction is determined using the BCA protein assay kit (Pierce™).

### 1.3) Analysis of Fractions by Dotblot

Ten μg of proteins are absorbed by capillary action onto a nitrocellulose membrane (Biorad apparatus) and processed for DotBlot analysis with anti-toxic oligomeric amyloid peptide (Aβo, E22P), anti-fibrillar amyloid peptide (Aβ-F, OC) and anti-Tau phosphorylated on Threonine 181(pTau^{Thr181} ) or Threonine 205 (pTau^{Thr205}) antibodies. Normalization of the results is carried out using an anti-GAPDH antibody or amido black staining for the soluble fraction or after Ponceau Red staining for the insoluble fraction.

### 2) Monitoring in the Brain and Intestines

Proteins and peptides are recovered from different tissues and organs

### Procedure

### 2.1) Dissection

At the different ages studied, mice were sacrificed by cervical dislocation. Brains and intestines from wild-type (WT) mice and APP/PS1 mice are collected. The brain is extracted from the braincase and dissected to separate the cortex and hippocampus. The intestines are recovered and compartmentalized as follows: duodenum, jejunum, ileum, and colon.

These samples are separated into 2 batches before being frozen (in liquid nitrogen then stored at −80° C., in OCT):
one of the batches will be used for microscopic analysis after preparation of a 30 μm section
the other batch will be used for biochemical experiments (Western Blot) or molecular biology experiments (QPCR).

### 2.2) Protein Extraction

Intestinal and brain samples are taken up in 1 ml of RIPA buffer (50 mM Tris-HCl pH 8, 150 mM NaCl, 1% NP-40, 0.5% sodium deoxycholate and 0.1% SDS) containing a cocktail of protease and phosphatase inhibitors. The tissues are ground using an automated program on a Miltenyi grinder. The homogenate is then centrifuged for 10 minutes at 4° C. at 2,500 rpm. The supernatant is recovered in a clean tube. It contains the proteins. The protein content of each fraction is determined using the BCA protein assay kit (Pierce ™).

### 2.3) Analysis of Lysates by Western Blot

Fifteen μg of proteins are run on an SDS-PAGE polyacrylamide gel (BioRad) at 100 volts for one hour and 30 minutes. The gel is then electrotransferred onto a polyvinylidene fluoride membrane (BioRad) and processed for analysis with anti-oligomeric amyloid peptide (Aβo, A11), anti-fibrillar amyloid peptide (Aβ-F, OC) and anti-Tau phosphorylated on Threonine 205(pTau^{Thr205} ) or Threonine 181 (pTau^{Thr181} ) antibodies.

### 2.4 Confocal Microscopy

Paraformaldehyde (PFA)-fixed tissues from WT and APP/PS1 mice were immersed in 20% sucrose in PBS overnight, then frozen in chilled PFA and stored at −30° C. Sections (20-30 μm thick) were cut with a cryostat microtome (HM 500M, Microm). The sections were blocked by incubation with phosphate-buffered saline (PBS) solution containing 0.1% (v/v) Triton X100 and 3% BSA for 30 min at room temperature. Tissue sections were then incubated overnight at 4° C. with primary antibodies directed against the various proteins of interest, washed 3 times with PBS containing 0.2% Tween 20 (PBS-T) and incubated for 2h at room temperature with secondary antibodies conjugated to different fluorochromes. After three washes in PBS-T, nuclei were stained by incubation in a Hoescht solution (1:200) for 5 minutes and the slides were mounted in a fluorescent mounting medium (Dako).

### 2.5) Monitoring in the Brain

The morphological study of spines and synaptic density is carried out by confocal microscopy using the Thy1-eYFP APP/PS1-21 mouse model. The distribution of Aβ and P-tau proteins is also analyzed by confocal microscopy with antibodies specific for the different proteins. The presence of amyloid plaques is detected by confocal microscopy using thioflavin-T. The expression levels of these different proteins are determined by Western Blot, DotBlot and/or Elisa.

### 2.6) Monitoring in the Intestines

As with the brain, the distribution of Aβ and P-tau proteins is analyzed by confocal microscopy with antibodies specific for the different proteins. This technique also makes it possible to understand the structure and morphology of intestinal villi and crypts. The expression levels of these different proteins are determined by Western Blot, DotBlot. The inflammatory context will be validated by QPCR and Elisa assays of different cytokines.

### 3) Results

Analysis of fecal Aβ peptides (toxic oligomeric E22P and prefibrillar OC forms by conformation-specific antibodies) and Tau-P^{Thr181} and Tau-P^{Thr205} proteins revealed a significant difference in the soluble fractions extracted from feces between WT and TG animals, as early as 1 month of age, before any detectable accumulation in the brain FIG. 1A. Indeed, transgenic mice eliminate significantly more toxic Aβo (E22P), pTau^{Thr181} and pTau^{Thr205} than WT animals, at 1 month of age. These differences persist at 2 months of age (results not shown). From 4 months of age FIG. 1B and FIG. 1C, the differences between WT and TG animals are less significant. At 9 months of age FIG. 1D, the difference in the elimination of toxic Aβo (E22P) and pTau^{Thr181} forms between WT and TG animals persists and increases. Similarly, at this age, a significant elimination of pre-fibrillar Aβ forms (OC) is observed in TG animals.

In the insoluble fractions, an increase in the detection of Tau-P^{Thr181}Tau-P^{Thr205} and Aβ-F (OC) proteins is observed in TG animals compared to WT animals at 4 months of age FIG. 2B. From 6 months of age, [FIG. 2C and FIG. 2D], WT animals eliminate pTau^{Thr181} identically, or even more pTau^{Thr205} or Aβ-F (OC) than transgenic animals.

In the brain, amyloid plaques are detected in the cortex and hippocampus of mice from 3 months of age. These observations are accompanied by morphological changes and a decrease in synapse density.

In the intestine, as early as 6 weeks of age, an accumulation of the toxic oligomeric form of Aβ (Aβo) and the pre-fibrillar form (Aβ-F) is observed. However, there is no amyloid plaques in the intestine. This accumulation is associated with a disorganization of the epithelial barrier. These observations suggest a modification of the barrier properties leading to an alteration of the nutrient absorption capacities and/or the transit motility of TG mice along with the development of an inflammatory context.

These results demonstrate that, unexpectedly, the gastrointestinal tract reflects what is happening in the brain with respect to Aβ and tau. According to the invention, robust molecular and histological changes are observed, heralding AD in the intestine, even before the onset of the first symptoms, i.e. before any brain involvement and specifically related to this disease.

The use of feces compared to blood or saliva markers has the advantage of being much earlier and more sensitive (the eliminated Aβ peptides and tau proteins are stable and present in significant quantities).

The assay of these markers in the stool therefore constitutes an effective tool for the early diagnosis of AD and monitoring the progression of the disease.

All of these observations indicate that intestinal changes follow the progression of AD in the brain. The intestine also reveals an accumulation of Aβ peptides and phosphorylated tau proteins in the feces, which was detected before any accumulation in the brain. Indeed, an accumulation and then elimination of soluble proteins associated with intestinal morphological changes, followed by clearance of insoluble proteins in the feces, were observed.

1.-17. (canceled)

18. A method for the in vitro diagnosis of a neurodegenerative disease, comprising the following steps
detecting the presence of at least one marker in a stool sample of an individual
the individual being a human or animal at an early stage of the neurodegenerative disease,
the marker is chosen from the derived forms of phosphorylated tau proteins selected from the hyperphosphorylated forms of the proteins, the aggregated forms of the proteins and the modified phosphorylated tau proteins resulting from one or several post-translational modifications.

19. The method according to claim 18, wherein the individual is asymptomatic.

20. The method according to claim 18, comprising the following steps:
breaking into contact the stool sample of the individual with at least one antibody or antibody portion specifically recognizing at least one of the markers,
forming a complex between the antibody or antibody portion and the marker,
detecting the presence of the complex.

21. The method according to claim 18, further comprising detecting, in the stool sample, the presence of derived forms of amyloid beta (Aβ) peptides selected from the oligomers of the peptides and the prefibrillar and fibrillar aggregated forms of the peptides.

22. The method according to claim 18, wherein the neurodegenerative disease is Alzheimer's disease.

23. A method for monitoring the condition of a human or animal individual suspected of suffering from a neurodegenerative disease, comprising the following steps:
breaking into contact a first stool sample of the individual with at least one antibody or antibody portion specifically recognizing at least one marker chosen from phosphorylated tau proteins and the derived forms of the proteins, and forming a complex between the antibody or antibody portion and the marker,
measuring the content of the complex in the first sample,
then taking a second stool sample of the individual, the second sample being taken after the first sample
breaking into contact the second stool sample with the antibody or antibody portion, and forming a complex between the antibody or antibody portion and the marker,
measuring the content of the complex in the second sample,
a variation between the contents of the complex in the first sample and of the complex in the second sample indicating a progression of the disease or a regression of the disease
a content of the complex in the second sample higher than the content of this same complex in the first sample indicates a progression of the disease, at an early stage,
a content of the complex in the second sample lower than the content of this same complex in the first sample indicates a regression of the disease.

24. The method according to claim 23, further comprising
breaking into contact the first stool sample of the individual with at least one antibody or antibody portion specifically recognizing at least one amyloid beta peptide or a derived form of a soluble amyloid beta peptide and forming a complex therewith,
measuring the content of the complex in the first sample,
breaking into contact the second stool sample of the individual with the antibody or antibody portion,
measuring the content of the complex in the second sample,
a variation between the contents of the complex in the first sample and of the complex in the second sample indicating a progression of the disease or a regression of the disease
a content of the complex in the second sample higher than the content of this same complex in the first sample indicates a progression of the disease, at an early stage, or
a content of the complex in the second sample lower than the content of this same complex in the first sample indicates a regression of the disease.

25. The method according to claim 23, wherein the individual is treated for the disease.

26. The method according to claim 23, wherein the neurodegenerative disease is Alzheimer's disease.

27. A method for screening a drug for treating a neurodegenerative disease, in an individual suspected of suffering from such a disease, comprising the following steps:
breaking into contact a first stool sample of the individual with at least one antibody or antibody portion specifically recognizing at least one marker chosen from phosphorylated tau proteins and the derived forms of the proteins, and forming a complex between the antibody or antibody portion and the marker,
measuring the content of the complex in the first sample,
then taking a second stool sample of the individual, the second sample being taken after the first sample
breaking into contact the second stool sample with the antibody or antibody portion, and forming a complex between the antibody or antibody portion and the marker,
measuring the content of the complex in the second sample,
a variation between the contents of the complex in the first sample and of the complex in the second sample indicating the effectiveness or ineffectiveness of the drug,
a content of the complex in the second sample lower than the content of this complex in the first sample indicates an effectiveness of the drug.

28. The method according to claim 27, further comprising
breaking into contact the first stool sample of the individual with at least one antibody or antibody portion specifically recognizing at least one amyloid beta peptide or a derived form of a soluble amyloid beta peptide and forming a complex therewith,
measuring the content of the complex in the first sample,
breaking into contact the second stool sample of the individual with the antibody or antibody portion and forming a complex therewith,
measuring the content of the complex in the second sample,
a variation between the contents of the complex in the first sample and of the complex in the second sample indicating the effectiveness or ineffectiveness of the drug,
the content of the complex in the second sample lower than the content of this complex in the first sample indicates an effectiveness of the drug

29. The method according to claim 27, wherein the neurodegenerative disease is Alzheimer's disease.

30. A kit for the in vitro detection of a neurodegenerative disease, at the early stage, of a human or animal individual, comprising
at least one antibody or antibody portion specifically recognizing at least one marker chosen from the derived forms of phosphorylated tau proteins selected from the hyperphosphorylated forms of the proteins, the aggregated forms of the proteins and the modified phosphorylated tau proteins resulting from one or several post-translational modifications, and
the means allowing the formation of a complex between the antibody or antibody portion and the marker.

31. The kit according to claim 30, further comprising at least one antibody or antibody portion specifically recognizing at least one marker chosen from the derived forms of amyloid beta (Aβ) peptides selected from the oligomers of the peptides and the prefibrillar and fibrillar aggregated forms of the peptides.
